(19) United States
(12) Patent Application Publication
(10) Pub. No.: US 2020/0187796 A1
(43) Pub. Date: Jun. 18, 2020

(54) Photoplethysmogram Detector

(71) Applicant: Intel Corporation, Santa Clara, CA (US)

(72) Inventors: Sean Jude William Lawrence, Bangalore (IN); Ayeshwarya B. Mahajan, Bangalore (IN)

(73) Assignee: Intel Corporation, Santa Clara, CA (US)

(21) Appl. No.: 16/796,617

(22) Filed: Feb. 20, 2020

(51) Int. Cl. A61B 5/026; A61B 5/00; A61B 5/1455; A61B 5/0205

(52) U.S. Cl. CPC A61B 5/0261; A61B 5/7235; A61B 5/6826; A61B 5/14552; A61B 5/02427; A61B 5/0205; A61B 2562/146; A61B 2562/0285; A61B 2562/185; A61B 5/6897

(57) ABSTRACT

There is disclosed in one example a photoplethysmogram (PPG) sensor, including: a light-emitting diode (LED); a photodetector including an electrically-biased graphene metal junction having a conductor running therethrough; a current sensor to sense a current from the photodetector; and processing logic to compute a blood volume flow rate from the sensed current.

### FIELD OF THE SPECIFICATION

This disclosure relates in general to the field of biometrics, and more particularly, though not exclusively, to a system and method for providing a photoplethysmogram detector.

### BACKGROUND

Biometrics are playing an increasingly important role in human-to-machine interfaces.

### BRIEF DESCRIPTION OF THE DRAWINGS

The present disclosure is best understood from the following detailed description when read with the accompanying FIGURES. It is emphasized that, in accordance with the standard practice in the industry, various features are not necessarily drawn to scale, and are used for illustration purposes only. Where a scale is shown, explicitly or implicitly, it provides only one illustrative example. In other embodiments, the dimensions of the various features may be arbitrarily increased or reduced for clarity of discussion.

FIG. 1 is a perspective view illustration of a human subject using a pulse oximeter.

FIG. 2 is a perspective view illustration of a gaming laptop.

FIG. 3 is a graph illustrating a typical photoplethysmogram (PPG) waveform.

FIGS. 4A and 4B illustrate an embodiment of a PPG sensor.

FIG. 5 is a flowchart of a method of using a PPG sensor to control a computing apparatus.

FIG. 6 is a block diagram illustration of a PPG sensor.

FIG. 7 is an additional block diagram of a PPG sensor.

FIG. 8 is a block diagram of a processor that may have more than one core, may have an integrated memory controller, and may have integrated graphics according to one or more examples of the present specification.

FIG. 9 is a block diagram of a multiprocessor system.

FIG. 10 is a block diagram of a system-on-a-chip (SoC).

### EMBODIMENTS OF THE DISCLOSURE

The following disclosure provides many different embodiments, or examples, for implementing different features of the present disclosure. Specific examples of components and arrangements are described below to simplify the present disclosure. These are, of course, merely examples and are not intended to be limiting. Further, the present disclosure may repeat reference numerals and/or letters in the various examples. This repetition is for the purpose of simplicity and clarity and does not in itself dictate a relationship between the various embodiments and/or configurations discussed. Different embodiments may have different advantages, and no particular advantage is necessarily required of any embodiment.

Biometrics play an increasing role in computing, and especially in mobile computing devices and laptops. For example, many devices now provide biometric authentication means, such as fingerprint sensors, retinal scans, and similar. These biometric sensors can be used to increase security and/or convenience. For example, a smartphone equipped with biometric authentication may not require a user to enter a personal identification number (PIN), or a password, but rather can authenticate the user by permitting the user to simply place a finger on a fingerprint sensor. To increase security, biometrics may be used as a second factor in a two-factor authentication scheme. For example, a user may be required to enter a password and use a fingerprint scanner.

Biometrics may also be used to improve the computing experience. For example, a computing device may integrate biometric sensors, such as photoplethysmogram (PPG) and/or electrocardiogram (ECG) sensors, to provide so-called “intentional sensing.” For example, sensors may be placed on palm rests, key caps, hinges, “A” and “C” laptop covers, in gaming headsets, or at other locations.

PPG is typically measured from a person's finger, or from the person's earlobe. An illustrative PPG sensor includes a light-emitting diode (LED) and a photodetector (PD), separated spatially in the same plane. The LED may be a red LED, and light incident from the LED on the finger may be modulated by absorption of the red light into the blood flow through the finger, earlobe, or other measurement point.

PPG measures blood volume flow, allowing for calculation of oxygen absorption or saturation on the principle that the higher the blood oxygen saturation (SpO2), the more red light is absorbed. Thus, the amount of light reflected back to the PD varies inversely with the SpO2. By measuring the light reflected, the PPG sensor can compute the SpO2 based on the volume of light absorbed.

Some existing PPG sensors have disadvantages, because a relatively large surface area is required to provide a coplanar LED and PD. They may also suffer losses resulting from light reflection and dispersion from the angular incidence of light between the LED and PD, for example, on the user's finger or earlobe. Because the LED and detector are optically isolated and spatially separated from one another, ambient light may also interfere with the reflected light.

There is disclosed herein an improved PPG sensor. At least some embodiments of the PPG sensor disclosed herein do not require the LED and the PD to be spatially separated on the same plane. Rather, these embodiments take advantage of the photoconductive properties of biased graphene, which allows for linear stacking of the transmitter and receiver on top of one another, thus reducing the amount of modulated light signal loss.

In an illustrative embodiment, a PPG sensor is made by stacking a film of graphene having disposed therein metal conductors or nanowires (such as silver nanowires) over a light-emitting diode (LED). The photoconductive properties of biased graphene metal junctions enable the graphene to be used as the PD in this configuration. The biased metal conductors conduct the changes in photo current in the graphene, due to the different amounts of light incident on and reflecting off of a person's finger falling on the graphene. These current changes could be amplified, sensed, and processed. The person's PPG may be measured as a voltage change on these lines, due to the amount of reflected light on the graphene around these lines.

In measuring light absorption (and thus, blood volume flow and oxygen saturation within the blood) the PD in this stacked configuration will have induced current from both the light transmission from the original LED pulse, and from light reflected back from the finger, or other point of measurement. There are disclosed herein methods for separating the current induced by the original light pulse from the reflected current that represents an indirect measure of blood volume flow and oxygen saturation. In a very simple embodiment, the system may be calibrated with the known induced current from the LED pulse. This can be subtracted from the overall current induced on the conductors within the graphene substrate to produce current induced by the reflected light. This embodiment has the advantage of being inexpensive, but is also less reliable, and is very subject to parameter drift in the solid state components often used in such sensors.

Another embodiment that is less susceptible to parameter drift relies on the principle that the induced current will have both a steady state (DC) component, and a time varying (AC) component. It should be noted here that, to avoid saturation of the graphene PD, the LED may be pulsed at some predetermined frequency. Thus, the graphene substrate will experience a time varying induced current based on those pulses. However, for the duration of the pulse, the induced current can be treated as a DC signal. Furthermore, the return pulse or reflection will experience a time invariant maximum reflection value, corresponding to the minimum flow volume of the blood. As the heart beats, the volume of blood at the measurement point changes, and thus, the measured reflection will change. The blood pressure will have a systolic maximum and a diastolic minimum. It is expected that maximum reflection (and thus, maximum current) will correspond with the diastolic minimum, while minimum reflection (and therefore, minimum current) will correspond with the systolic maximum.

A low-pass filter can be used to isolate the DC baseline (representing the sum of the current induced by the outgoing light pulse and the baseline minimum absorption from the desired time variant signal). This effectively isolates the time varying component of the reflected light from the relatively time invariant outgoing light pulse and the DC baseline of the reflected light pulse. This permits an accurate measurement of blood volume flow, even in the presence of these extra signals.

Advantageously, by removing the optical isolation between the LED and the PD, embodiments disclosed herein increase the surface area of the finger or other sense point relative to the detection mechanism. This improves the fidelity of the PPG signal. Furthermore, by reducing the size of the sensor, there is less interference from ambient light.

Smaller sensors enable newer use cases and measurement points, because of their significantly smaller spatial footprint. For example, there is a trend in laptops toward smaller form factors, and especially toward smaller bezels, which from the user's perspective feel like wasted space. Laptops are also becoming thinner and lighter. Thus, the available space for extra sensors—even in high-end gaming laptops and other top-of-the-line devices—continues to shrink.

Intentional PPG is a beneficial sensing mechanism for intentional wellness on some next-generation computing form factors. For example, these types of sensors may be used in next-generation “cognitive boost” features, available in some high-end products such as the Intel® vPro® platform. In an illustrative use case, a high-end gaming laptop includes PPG sensors that can detect the user's health state. For example, if the user is playing a first-person shooter, the user's stress level may correlate to blood flow rate, SpO2, and/or pulse, among other biometric factors. As the user's pulse increases and/or the blood flow rate correspondingly decreases, it may be inferred that the user is experiencing more stress. If the user is playing a first-person shooter (FPS) game, the application may include code that responds to the user's state. If the user is becoming very stressed by the game, then the intensity of the game can be decreased to help the user avoid health risks associated with hyperventilation or increased stress. On the other hand, if the user is playing a game that should be exciting to an appropriate level, and the user's pulse is very low, this may indicate that the user is bored. For example, it may be a game that the user has played multiple times and knows well. In that case, the intensity of the game can be increased (e.g., by providing the user more adversaries and/or more obstacles, more quickly) to increase the user's enjoyment of the game, even upon multiple replays. In other words, a game can be programmed to automatically adjust its own difficulty based on the user's response to the game.

PPG sensors according to the present specification can be placed on a mouse trackpad, on the A or C shells of a clamshell laptop design, on hinges, on keys of the keyboard (e.g., on the space bar, or on keys commonly used for movement such as the arrow keys, or other commonly used keys such as ASD, ZXC, QWE, or others), or at other places where the user is expected to frequently interact with the device.

In the case of a remote sensor (e.g., such as in a gaming headset), the data may be transmitted wirelessly, such as via Bluetooth. In these cases, processing may occur on the device itself (e.g., within the user's headset), or raw data can be transmitted to the host machine and processing can occur there.

A system and method for providing a photoplethysmogram detector will now be described with more particular reference to the attached FIGURES. It should be noted that throughout the FIGURES, certain reference numerals may be repeated to indicate that a particular device or block is wholly or substantially consistent across the FIGURES. This is not, however, intended to imply any particular relationship between the various embodiments disclosed. In certain examples, a genus of elements may be referred to by a particular reference numeral (“widget 10”), while individual species or examples of the genus may be referred to by a hyphenated numeral (“first specific widget 10-1” and “second specific widget 10-2”).

Certain of the FIGURES below detail example architectures and systems to implement embodiments of the above. In some embodiments, one or more hardware components and/or instructions described above are emulated as detailed below, or implemented as software modules.

In certain examples, instruction(s) may be embodied in a “generic vector-friendly instruction format.” In other embodiments, another instruction format is used. The description below of the write mask registers, various data transformations (swizzle, broadcast, etc.), addressing, etc. is generally applicable to the description of the embodiments of the instruction(s) above. Additionally, example systems, architectures, and pipelines are detailed below. Embodiments of the instruction(s) above may be executed on those systems, architectures, and pipelines, but are not limited to those detailed.

An instruction set may include one or more instruction formats. A given instruction format may define various fields (e.g., number of bits, location of bits) to specify, among other things, the operation to be performed (e.g., opcode) and the operand(s) on which that operation is to be performed and/or other data field(s) (e.g., mask). Some instruction formats are further broken down though the definition of instruction templates (or subformats). For example, the instruction templates of a given instruction format may be defined to have different subsets of the instruction format's fields (the included fields are typically in the same order, but at least some have different bit positions because there are fewer fields included) and/or defined to have a given field interpreted differently. Thus, each instruction of an instruction set architecture (ISA) is expressed using a given instruction format (and, if defined, in a given one of the instruction templates of that instruction format) and includes fields for specifying the operation and the operands. In one embodiment, an example ADD instruction has a specific opcode and an instruction format that includes an opcode field to specify that opcode and operand fields to select operands (source1/destination and source2); and an occurrence of this ADD instruction in an instruction stream will have specific contents in the operand fields that select specific operands. A set of SIMD extensions referred to as the advanced vector extensions (AVXs) (AVX1 and AVX2), and using the vector extensions (VEX) coding scheme has been released and/or published (e.g., see Intel® 64 and IA-32 Architectures Software Developer's Manual, September 2014; and see Intel® Advanced Vector Extensions Programming Reference, October 2014).

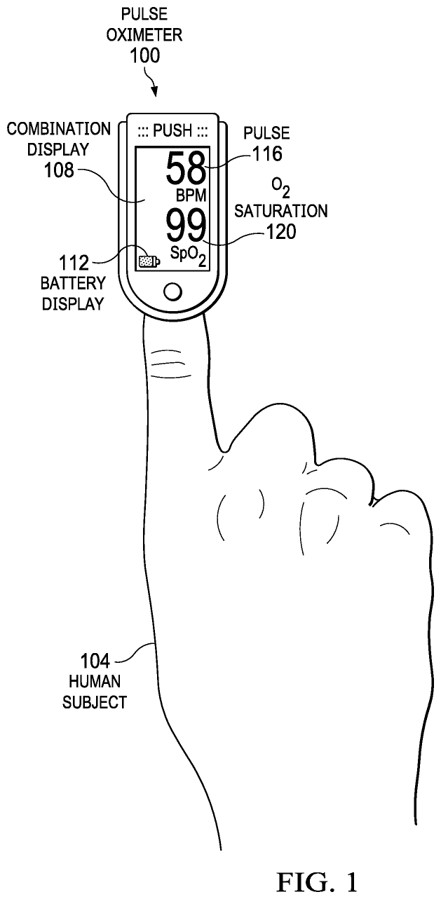

FIG. 1 is a perspective view illustration of a human subject 104 using a pulse oximeter 100. In this case, pulse oximeter 100 is a small, portable, battery-operated device. Pulse oximeter 100 clips over a finger of human subject 104, commonly the index finger. Pulse oximeter 104 includes a combination display 108 that displays both pulse 116 and 02 saturation 120, as measured by a PPG sensor. In addition to O2 saturation, the PPG sensor can be used to measure factors such as pulse and heart rate. Combination display 108 also includes a battery display 112 to indicate the battery charge available for pulse oximeter 100.

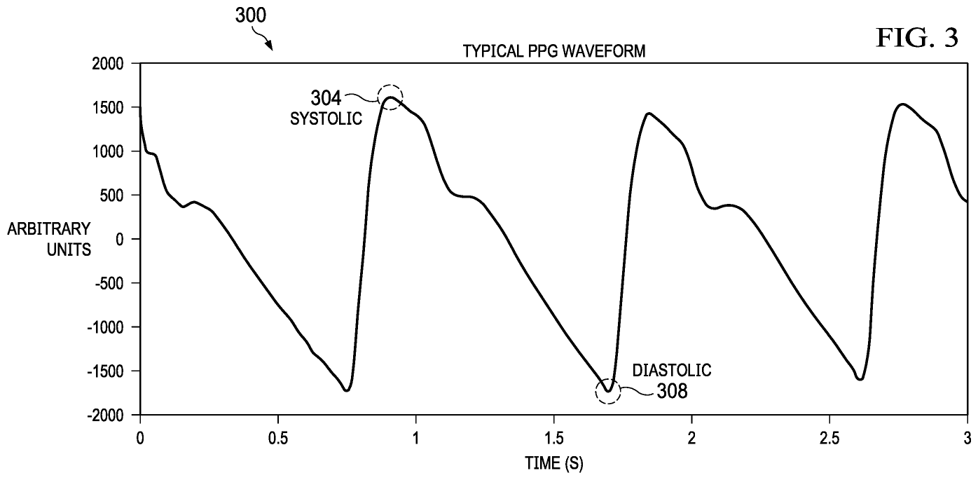

As illustrated here, PPG can be measured by illuminating the skin and measuring the changes in light absorption. This may typically be done by monitoring the perfusion of blood to the dermis and subcutaneous tissue of the skin. An LED transmits light to the skin, and the reflected light is measured by a photodiode. The alternating current (AC) component may be directly attributable to variations in blood volume in the skin caused by the pressure pulse of the cardiac cycle. Thus, each cardiac cycle may be represented by a PPG wave with a crest and a trough. A typical waveform is illustrated in graph 300 of FIG. 3.

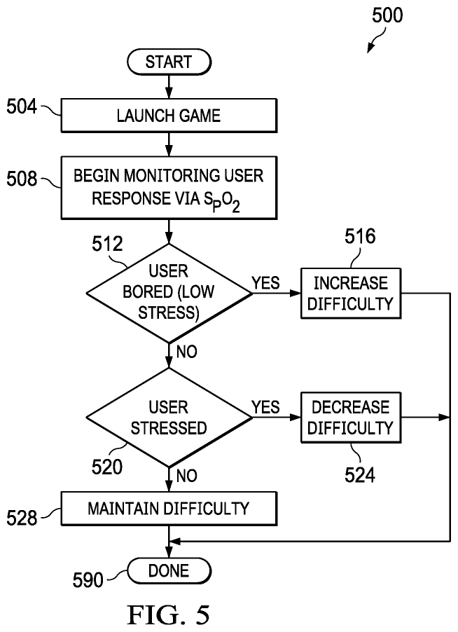

Pulse oximeter 100 may use a traditional pulse oximeter, using an optically isolated photodiode as a photodetector. However, pulse oximeter 100 could also be modified to use a graphene photodetector, as illustrated in FIG. 6. When a graphene photodetector is used, pulse oximeter 100 could be thinner, lighter, and may consume less battery power.

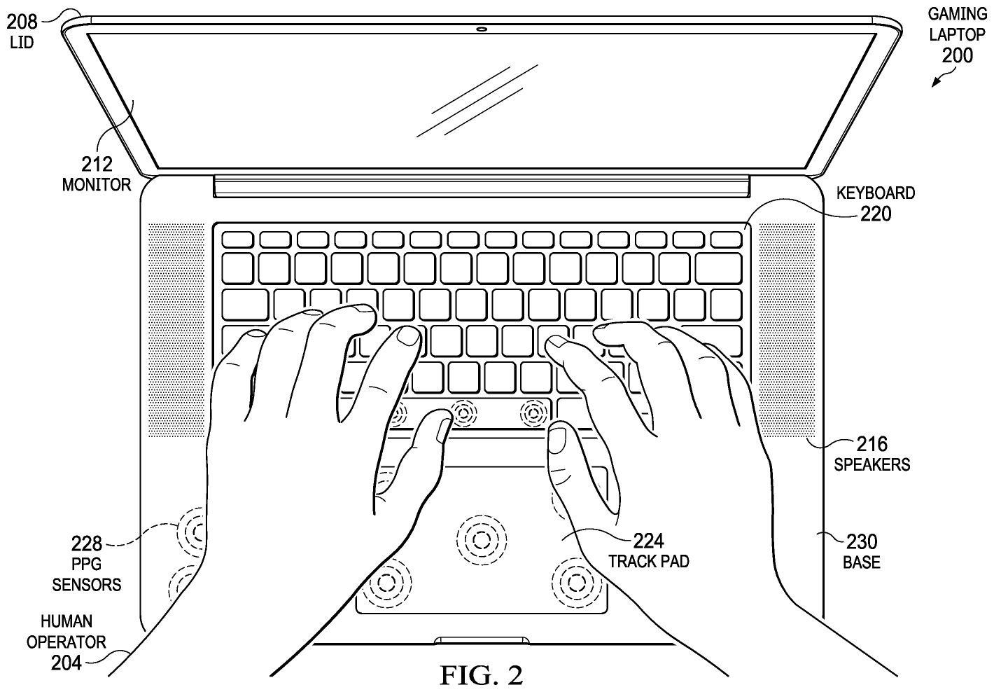

FIG. 2 is a perspective view illustration of a gaming laptop 200. Laptop 200 may be a gaming laptop, or any other suitable laptop. Laptop 200 is illustrated in this view as a gaming laptop to illustrate a common application for a PPG sensor in a laptop computer.

Some contemporaneous, high-end gaming laptops include one or more PPG sensors that can be used for intentional PPG to adjust the user's gaming experience, according to the stress that the user experiences. For example, if the PPG sensor indicates that the user's blood flow rate, SpO2 and/or other indicators such as pulse and heart rate as suited to a particular embodiment have dropped below normal, this may indicate that the user is stressed. If the user is stressed, this may indicate that the current difficulty of the game that the user is playing is excessive. In an extreme case, this could be a health concern. For example, if the user becomes extremely stressed, the user could experience cardiac arrest or other ill health effects. But even in a case where the user does not experience severe health effects from the difficulty of the game, the user may become frustrated, and may no longer enjoy playing the game. This at least has commercial implications for whether the user will continue to pay for paid content with the game, recommend the game to friends, or buy other games from the same studio.

On the other hand, if the measured blood flow rate, SpO2, and/or heart rate and pulse values provided by a PPG sensor indicate that the user is bored, this could also have commercial implications for the game. Users tend to not want to play boring games. Thus, some games are provided with adaptive difficulty settings, so that the difficulty of the game automatically adjusts to compensate for the user's present gaming experience. If the PPG sensors indicate very high stress, the game difficulty may be slackened to reduce the user's stress level. On the other hand, if the PPG sensor indicates boredom, the game difficulty may be increased to enhance user engagement. In one illustrative example, an optimal difficulty setting is one that causes the user to experience slightly increased stress, indicating engagement with the game, without reaching extreme stress. This may indicate that the user is engaging with and enjoying the game, but not becoming frustrated or angry.

To provide these functions, gaming laptop 200 includes traditional facilities such as a monitor 212, a keyboard 220, speakers 216, and a trackpad 224. These enable human operator 204 to interact with gaming laptop 200, including with the game, itself. These facilities provide both user input and machine output.

In this example, gaming laptop 200 is provided in a clamshell form factor, including a lid 208 (the so-called “A” surface of the clamshell). Lid 208 may be hingedly connected to base 230 (the so-called “C” surface of the clamshell) at a hinge point.

In this example, one or more PPG sensors 228 may be located on base 230 of gaming laptop 200.

In this illustration, a plurality of PPG sensors 228 are illustrated, but in other embodiments, as few as one PPG sensor may be provided. The location of the PPG sensor may vary, depending on the use case. For example, a PPG sensor could be located on a wrist rest of base 230 to take PPG measurements from the user's wrist. In other cases, PPG sensors could be located on trackpad 224, or on selected keys of keyboard 220.

Note that for effective monitoring, PPG sensors need not necessarily provide a continuous stream of PPG measurement data. Rather, occasional sampling of the PPG may be sufficient to track the user's health state over a period of minutes or hours. Indeed, being overly reactive to PPG measurements may, in fact, reduce the enjoyability of a game. For example, a user's heart rate may increase momentarily as the user engages in a particularly exciting part of a game, such as a “boss battle.” This is intended and desirable, and making the boss battle too easy responsive to this stimulus may be nonoptimal. However, if the boss battle is taking a very long time and the user is experiencing extreme stress, then it may be desirable to reduce the difficulty.

In some cases, when PPG sensors are placed on keys of keyboard 220, keys commonly used for gaming operations may be selected. For example, the arrow keys, space bar, W, A, S, D, F, Z, X, C, enter, control, alt, and shift keys may all be examples of keys commonly used in gaming operations. Placing a PPG sensor on one of these keys helps to ensure that measurements can be taken, even if they are not necessarily continuously taken.

FIG. 3 is a graph 300 illustrating a typical photoplethysmogram (PPG) waveform. In graph 300, arbitrary units of absorption are on a scale between −2000 and 2000, with a time varying signal. In this example, the time varying signal spans approximately three seconds. The crests 304 represent the systolic portion of the cycle, while troughs 308 represent the diastolic portion of the cycle. In other words, on the systolic cycle, the heart pumps out blood, increasing the blood pressure, and in the diastolic cycle, the heart rest between pulses, thus resulting in a region of lower blood pressure. The crests and troughs of the graph can be compensated for in a PPG sensor, for example, by using a low-pass filter.

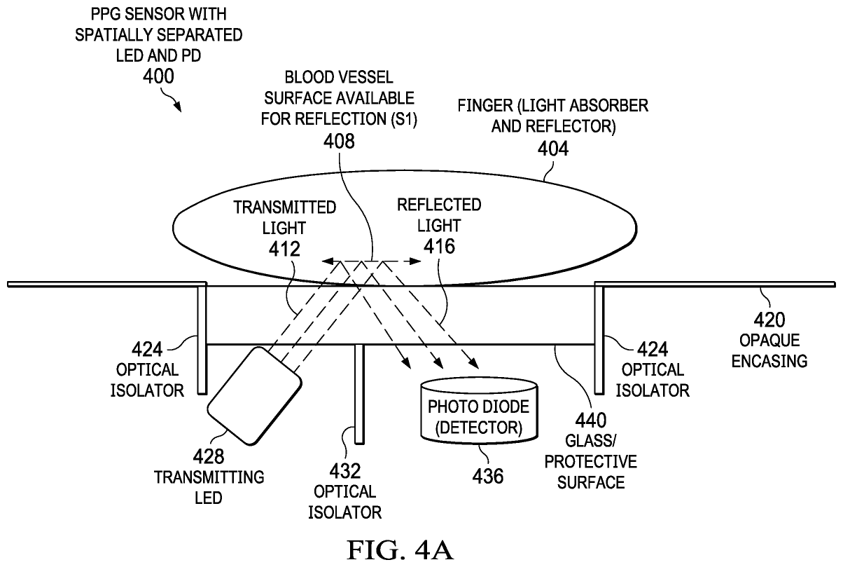

FIGS. 4A and 4B illustrate an embodiment of a PPG sensor 400. PPG sensor 400 uses a traditional optically isolated photodiode detector (PD) along with the LED transmitter.

PPG transmitter 400 includes a transmitting LED 428 that is optically isolated from a PD 436 by optical isolator 432. Also illustrated in this FIGURE are opaque encasing 420, optical isolators 424 forming sidewalls around a trough of PPG sensor 400, and a glass or protective surface 440.

When a light absorber and reflector, such as a human subject's finger 404, is placed over the cavity, then transmitted light 412 strikes a blood vessel 408 with a surface available for reflection. Reflected light 416 then strikes PD 436.

LED 428 transmits the desired wavelength (typically a green color) of light onto finger 404 when it is placed over the cavity. A portion of transmitted light 412 is reflected back as reflected light 416. This is reflected off of blood vessel 408 after being modulated with the PPG signal. The modulated, reflected light is detected by PD 436. The detected signal may then be processed to extract the PPG signal.

The spatial separation between LED transmitter 428 and PD 436 provides a greater width for the overall device. As illustrated in FIG. 4B, interfering ambient light 450 may have the opportunity to enter the cavity, and thus interfere with the light reflected onto photodiode 436. This can cause corruption in the PPG signal.

This configuration may also introduce optical noise. This noise may be due to light reflected from non-pulsatile physiological material such as skin, muscle, tendons, and similar. When the light is incident at any angle, the contribution of this component is higher, because of the nonuniformity of these materials. This may be exacerbated by the long path the light takes before being reflected. Accordingly, the light is modulated.

Furthermore, because of the spatial separation of the LED and the PD, light is incident at an angle on the skin and blood vessel surface. This reduces the available surface area of the blood vessels for reflection for stable PPG modulation. Designs may need to be concerned with incorporating elements to minimize ambient light to the PD by using smaller surface areas for finger placement, and optical isolation. This can reduce the fidelity of the desired signal.

To obtain an improved PPG signal, it is desirable to reduce the spatial separation between the LED and the photodetector, reduce the optical noise due to reflected light from non-pulsatile physiological material, and maximize the available surface area of the blood vessel for reflection. Note that out of the transmitted incident light onto the skin, only a small fraction of photons return to the sensor. And of the total photons collected, on the order of 1/100th or 1/1000th of them are modulated by heart-pumped blood flow.

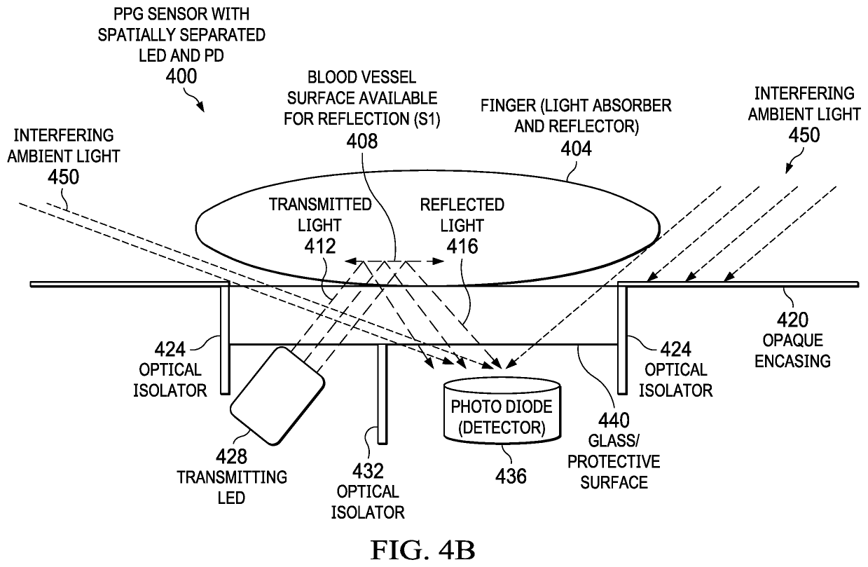

Embodiments of a PPG sensor as illustrated in FIG. 6, and as used for example in method 500 of FIG. 5, may minimize these undesirable factors.

FIG. 5 is a flowchart of a method 500 of using a PPG sensor to control a computing apparatus.

Method 500 illustrates a use case of a PPG sensor in which the PPG sensor is used to modify control of a game. Method 500 may be appropriately modified to control other aspects of the use of a laptop computer. For example, a PPG sensor could be used as part of an authentication scheme, or as part of a health monitoring service.

Starting in block 504, a user launches a game on a gaming laptop such as gaming laptop 200 of FIG. 2.

In block 508, the laptop begins monitoring the user response, via a PPG sensor. The PPG sensor may be used to monitor blood flow rate, SpO2, and/or heart rate as suited to a particular embodiment, and to make inferences about the user's current health or stress rate based on the PPG sensor.

In the example use case of a game, in decision block 512, the laptop determines whether the user is bored with the game. For example, if the user's heart rate is very low, this may indicate that the user is bored with the game, and thus may not be optimally enjoying the game. In this case, in block 516, the difficulty of the game may be increased to enhance the user's enjoyment.

Returning to decision block 512, if the user is not bored, then in block 520, the system may determine whether the user is stressed. For example, if the user has experienced an increase in heart rate over an extended period of time, or if the user has experienced a severe increase in heart rate over a short period of time, the system may infer that the user is stressed. In this case, in block 524, the system may decrease the difficulty of the game.

Returning to decision block 520, if the user is neither bored nor stressed, then in block 528, the current or baseline difficulty of the game may be maintained, on the assumption that the user is appropriately enjoying the game.

In block 590, the method is done.

FIG. 6 is a block diagram illustration of a PPG sensor 600. PPG sensor 600 includes a stacked LED and photodetector.

The stacked LED and photodetector minimizes the issues described above.

In this illustration, PPG sensor 600 is similar in many respects to PPG sensor 400 of FIGS. 4A and 4B.

PPG sensor 600 includes a transmitting LED 628, an optical isolator 624 forming a sidewall, a glass or protective surface 640, and an opaque casing 620. A human subject may place a finger 604 over protective surface 640, including a finger surface available for reflection (S2) 608. Transmitter LED 628 transmits transmitted light 612, which is reflected back as reflected light 616.

PPG sensor 600 also includes a photodetector, but in this case, the photodetector is a graphene sheet with silver nanowire conductors. Because transmitter 628 and photodetector 636 are stacked, protective surface 640 can form a narrower gap, thus not admitting ambient light 650. This helps to ensure that ambient light 650 cannot easily interfere with the signal. Furthermore, because transmitting LED 628 is stacked with graphene conductor 636, the incident angle of the transmitted light is also much lower.

Photodetector 636 may be a thin, transparent layer of graphene, with fine wires or other conductors. These may be, for example, silver nanowires. By applying a bias between these metal conductors, graphene-based photodiodes can be created.

The electrically-biased graphene sheet creates a transparent photodetector. When finger 604 is placed over the sensor, part of transmitted light 612 is reflected back as reflected light 616. The transmitted light creates a constant current in the conductive lines, due to the photoconductivity of the graphene metal junctions. The reflected component also creates a similar current component that contains a constant component and a variable component that corresponds to the user's PPG. Thus, the current in the conductors equals the photo current due to constant transmitive component, plus photo current due to constant reflected component, plus photo current variations due to PPG.

The PPG signal may be extracted by removing the constant components, such as via low-pass filtering. This signal may then be amplified to obtain the PPG value.

Positioning photodetector 636 over transmitter 628 addresses some of the fidelity issues discussed above.

For example, there is no lateral spatial separation between the LED and the PD. This can minimize or eliminate interference with reflected light carrying the PPG signal, provided there is a well-designed optical isolation implementation, as illustrated in FIG. 6.

When light is incident directly, or nearly directly, on the subcutaneous tissue, the contribution of the various components of the reflected light due to the non-pulsatile physiological material (e.g., skin, muscle, tendons, etc.) is less. The nonuniformity of these materials is also less, because of the shorter path the light travels before reflection. The light is therefore modulated, accordingly.

Furthermore, because of the stacking of the LED and the PD over each other, the available surface area of blood vessels for reflection for stable PPG modulation is maximized. This can be seen in FIG. 6, where S2 is noticeably and obviously greater than S1 of FIG. 4A.

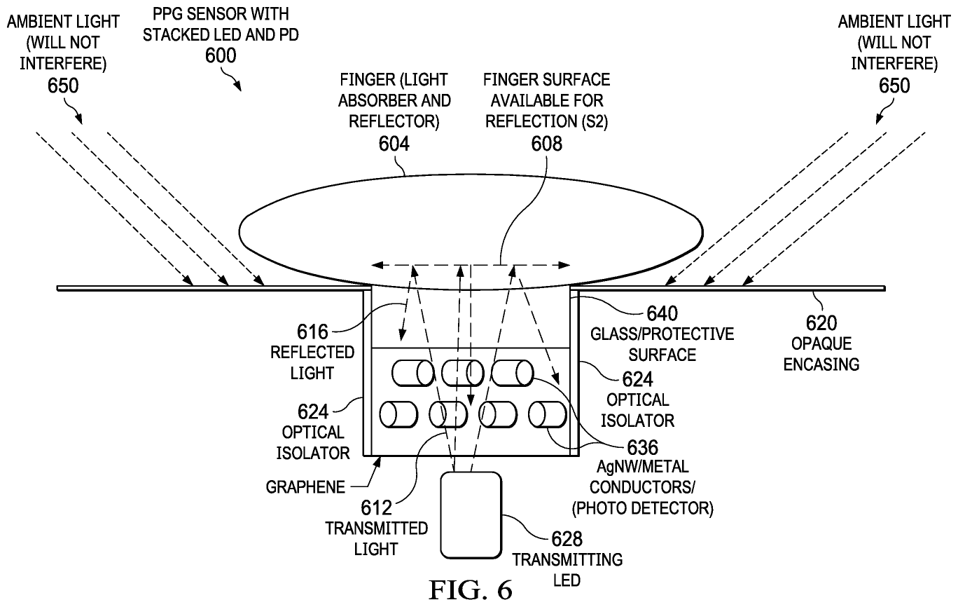

FIG. 7 is an additional block diagram of a PPG sensor 700. PPG sensor 700 may be a pulse oximeter, such as pulse oximeter 100 of FIG. 1, or the various PPG sensors within a gaming laptop 200 of FIG. 2, or other high-end laptop computer.

PPG sensor 700 includes a power source 708 that provides power to various components.

An LED transmitter 704 receives power from power source 708, and transmits light at a selected wavelength, such as green light.

A bias circuit 710 receives power from power source 708, and includes circuitry to bias conductors of photodetector 712. Photodetector 712 may be, for example, a graphene sheet with conductors running therethrough, such as silver nanowires. In some cases, photodetector 712 may be overlaid on LED 704, thus eliminating lateral spatial separation.

Photodetector 712 receives light reflected off of a surface such as a finger, including blood vessels available for reflection.

Current sensor 716 senses current within photodetector 712, which current may be induced by light received by the graphene sheet with the silver nanowires. This current may have a time invariant value caused by light transmitted by LED 704, a time invariant value from the reflected light, and a time varying value representing the PPG data.

To filter out the time invariant components of the sensed current, filter 720 may be a low-pass filter. This filters out the non-desired components to yield only the components that represent the PPG signal.

Optionally, amplifier 722 may amplify the filtered signal to provide ease in processing.

Processing logic 724 may include circuitry, and/or software or firmware, that can be used to process the signal. For example, processing logic may be used to implement method 500 of FIG. 5, or other methods consistent with this specification. Processing logic 724 may be used to display the sensed blood flow rate, SpO2, and/or pulse values as suited to a particular embodiment, as in the case of a standalone pulse oximeter 100 of FIG. 1. Alternatively, in the case of gaming laptop 200 of FIG. 2, or other similar embodiments, processing logic 724 may include both logic to sense and condition the signals, and logic to respond to those signals by altering the operation of the computer, itself. This could include, for example, monitoring a user's health, providing authentication, or altering the difficulty of a game, by way of illustrative and nonlimiting example.

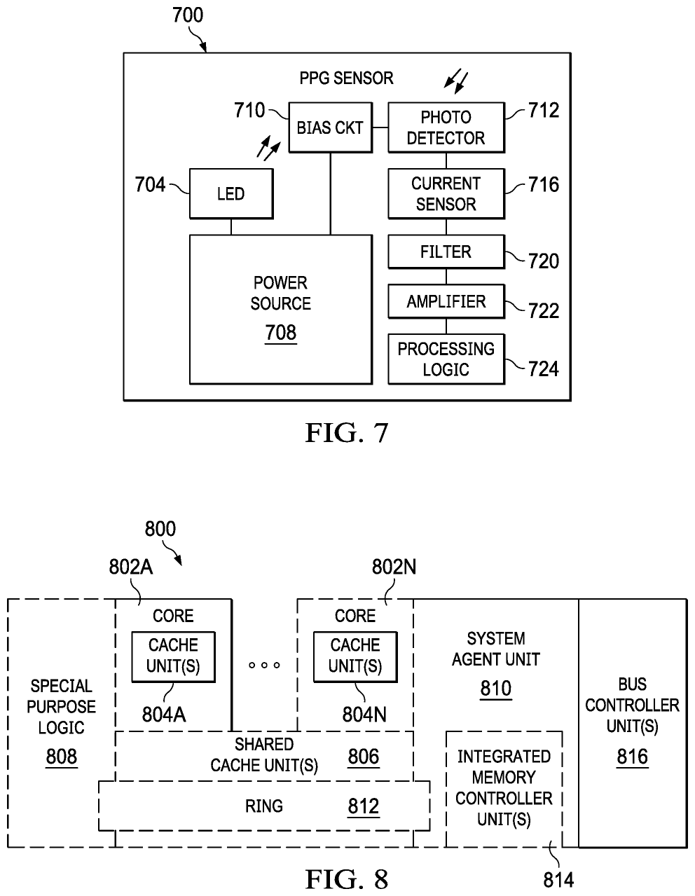

FIG. 8 is a block diagram of a processor 800 that may have more than one core, may have an integrated memory controller, and may have integrated graphics according to embodiments of the specification. Processor 800 may be configured or adapted to provide a PPG detector, as disclosed in the present specification.

The solid lined boxes in FIG. 8 illustrate a processor 800 with a single core 802A, a system agent 810, a set of one or more bus controller units 816, while the optional addition of the dashed lined boxes illustrates an alternative processor 800 with multiple cores 802A-N, cache units 804A-N, a set of one or more integrated memory controller unit(s) 814 in the system agent unit 810, and special-purpose logic 808.

Thus, different implementations of the processor 800 may include: 1) a central processing unit (CPU) with the special-purpose logic 808 being integrated graphics and/or scientific (throughput) logic (which may include one or more cores), and the cores 802A-N being one or more general purpose cores (e.g., general purpose in-order cores, general purpose out-of-order cores, a combination of the two); 2) a coprocessor with the cores 802A-N being a large number of special-purpose cores intended primarily for graphics and/or scientific throughput; and 3) a coprocessor with the cores 802A-N being a large number of general purpose in-order cores. Thus, the processor 800 may be a general purpose processor, coprocessor or special-purpose processor, such as, for example, a network or communication processor, compression engine, graphics processor, GPGPU, a high-throughput many integrated core (MIC) coprocessor (including 30 or more cores), embedded processor, or the like. The processor may be implemented on one or more chips. The processor 800 may be a part of and/or may be implemented on one or more substrates using any of a number of process technologies, such as, for example, BiCMOS, CMOS, or NMOS.

The memory hierarchy includes one or more levels of cache within the cores, a set or one or more shared cache units 806, and external memory (not shown) coupled to the set of integrated memory controller units 814. The set of shared cache units 806 may include one or more mid-level caches, such as level 2 (L2), level 3 (L3), level 4 (L4), or other levels of cache, a last level cache (LLC), and/or combinations thereof. While in one embodiment a ring based interconnect unit 812 interconnects the integrated graphics logic 808, the set of shared cache units 806, and the system agent unit 810/integrated memory controller unit(s) 814, alternative embodiments may use any number of well-known techniques for interconnecting such units. In one embodiment, coherency is maintained between one or more cache units 806 and cores 802A-N.

In some embodiments, one or more of the cores 802A-N are capable of multithreading. The system agent 810 includes those components coordinating and operating cores 802A-N. The system agent unit 810 may include, for example, a power control unit (PCU) and a display unit. The PCU may be or include logic and components needed for regulating the power state of the cores 802A-N and the integrated graphics logic 808. The display unit is for driving one or more externally connected displays.

The cores 802A-N may be homogenous or heterogeneous in terms of architecture instruction set; that is, two or more of the cores 802A-N may be capable of executing the same instruction set, while others may be capable of executing only a subset of that instruction set or a different instruction set.

Example Computer Architectures

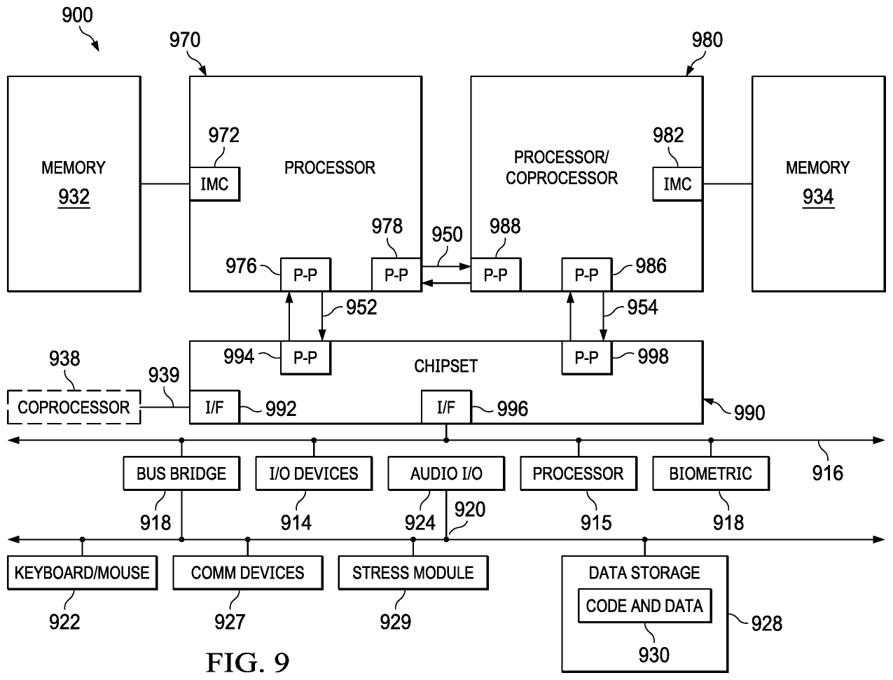

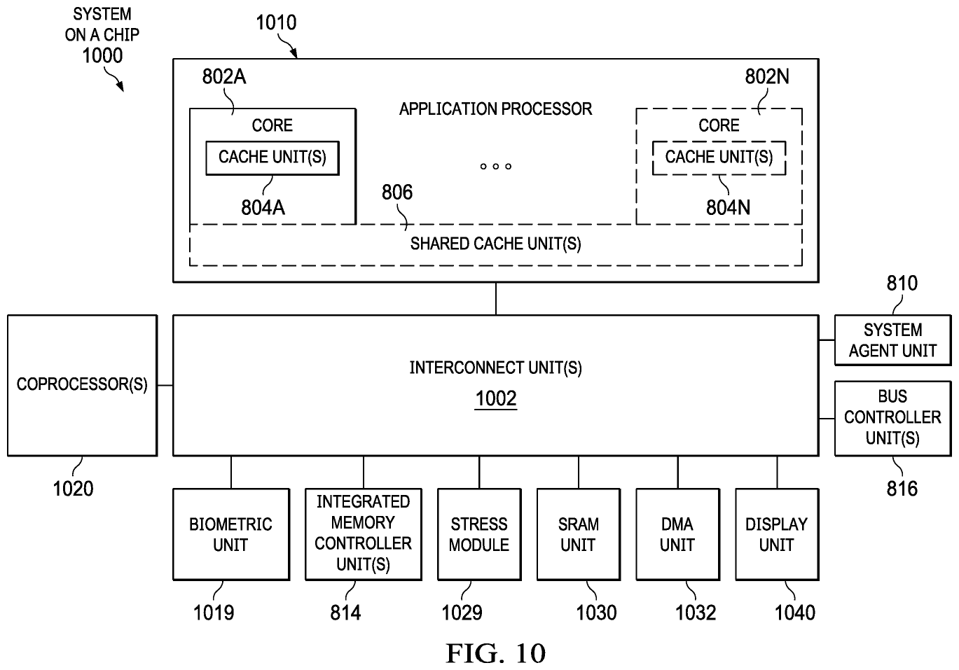

FIGS. 9 and 10 are block diagrams of example computer architectures. Other system designs and configurations known in the arts for laptops, desktops, handheld PCs, personal digital assistants, engineering workstations, servers, network devices, network hubs, switches, embedded processors, digital signal processors (DSPs), graphics devices, video game devices, set-top boxes, micro controllers, cell phones, portable media players, hand held devices, and various other electronic devices, are also suitable. In general, a huge variety of systems or electronic devices capable of incorporating a processor and/or other execution logic as disclosed herein are generally suitable.

FIG. 9 is a block diagram of a multiprocessor system 900. Multiprocessor system 900 may be configured or adapted to provide a PPG detector, as disclosed in the present specification.

As shown in FIG. 9, multiprocessor system 900 is a point-to-point interconnect system, and includes a first processor 970 and a second processor 980 coupled via a point-to-point interconnect 950. Each of processors 970 and 980 may be some version of the processor 800.

Processors 970 and 980 are shown including integrated memory controller (IMC) units 972 and 982, respectively. Processor 970 also includes as part of its bus controller units point-to-point (P-P) interfaces 976 and 978; similarly, second processor 980 includes P-P interfaces 986 and 988. Processors 970, 980 may exchange information via a point-to-point (P-P) interface 950 using P-P interface circuits 978, 988. As shown in FIG. 9, IMCs 972 and 982 couple the processors to respective memories, namely a memory 932 and a memory 934, which may be portions of main memory locally attached to the respective processors.

Processors 970, 980 may each exchange information with a chipset 990 via individual P-P interfaces 952, 954 using point-to-point interface circuits 976, 994, 986, 998. Chipset 990 may optionally exchange information with the coprocessor 938 via a high performance interface 939. In one embodiment, the coprocessor 938 is a special-purpose processor, such as, for example, a high-throughput MIC processor, a network or communication processor, compression engine, graphics processor, GPGPU, embedded processor, or the like.

A shared cache (not shown) may be included in either processor or outside of both processors, yet connected with the processors via P-P interconnect, such that either or both processors' local cache information may be stored in the shared cache if a processor is placed into a low power mode.

Chipset 990 may be coupled to a first bus 916 via an interface 996. In one embodiment, first bus 916 may be a peripheral component interconnect (PCI) bus, or a bus such as a PCI Express bus or another third generation IO interconnect bus, by way of nonlimiting example.

As shown in FIG. 9, various IO devices 914 may be coupled to first bus 916, along with a bus bridge 918 which couples first bus 916 to a second bus 920. In one embodiment, one or more additional processor(s) 915, such as coprocessors, high-throughput MIC processors, GPGPUs, accelerators (such as, e.g., graphics accelerators or DSP units), field programmable gate arrays, or any other processor, are coupled to first bus 916. In one embodiment, second bus 920 may be a low pin count (LPC) bus. Various devices may be coupled to a second bus 920 including, for example, a keyboard and/or mouse 922, communication devices 927 and a storage unit 928 such as a disk drive or other mass storage device which may include instructions or code and data 930, in one embodiment.

A stress module 929 may also be coupled to second bus 920, as illustrated. Stress module 929 could include a PPG sensor such as PPG sensor 600 of FIG. 6, along with associated processing logic to detect a user's stress level, and to respond accordingly. Further, an audio IO 924 may be coupled to the second bus 920. Note that other architectures are possible. For example, instead of the point-to-point architecture of FIG. 9, a system may implement a multidrop bus or other such architecture.

FIG. 10 is a block diagram of a system-on-a-chip (SoC) 1000. SoC 1000 may be configured or adapted to provide a PPG detector, as disclosed in the present specification.

Similar elements in FIG. 8 bear like reference numerals. Also, dashed lined boxes are optional features on more advanced SoCs. In FIG. 10, an interconnect unit(s) 1002 is coupled to: an application processor 1010 which includes a set of one or more cores 802A-N and shared cache unit(s) 806; a system agent unit 810; a bus controller unit(s) 816; IMC unit(s) 814; a set of one or more coprocessors 1020 which may include integrated graphics logic, an image processor, an audio processor, and a video processor; a stress module 1029 (which could include a PPG sensor such as PPG sensor 600 of FIG. 6, along with associated processing logic to detect a user's stress level, and to respond accordingly); a static random access memory (SRAM) unit 1030; a direct memory access (DMA) unit 1032; and a display unit 1040 for coupling to one or more external displays. In one embodiment, the coprocessor(s) 1020 includes a special-purpose processor, such as, for example, a network or communication processor, compression engine, GPGPU, a high-throughput MIC processor, embedded processor, or the like.

Embodiments of the mechanisms disclosed herein may be implemented in hardware, software, firmware, or a combination of such implementation approaches. Some embodiments may be implemented as computer programs or program code executing on programmable systems comprising at least one processor, a storage system (including volatile and nonvolatile memory and/or storage elements), at least one input device, and at least one output device.

Program code, such as code 930 illustrated in FIG. 9, may be applied to input instructions to perform the functions described herein and generate output information. The output information may be applied to one or more output devices, in known fashion. For purposes of this application, a processing system includes any system that has a processor, such as, for example, a DSP, a microcontroller, an application-specific integrated circuit (ASIC), or a microprocessor.

The program code may be implemented in a high level procedural or object oriented programming language to communicate with a processing system. The program code may also be implemented in assembly or machine language, if desired. In fact, the mechanisms described herein are not limited in scope to any particular programming language. In any case, the language may be a compiled or interpreted language.

One or more aspects of at least one embodiment may be implemented by representative instructions stored on a machine-readable medium which represents various logic within the processor, which when read by a machine causes the machine to fabricate logic to perform the techniques described herein. Such representations, known as “intellectual property (IP) cores” may be stored on a tangible, machine-readable medium and supplied to various customers or manufacturing facilities to load into the fabrication machines that actually make the logic or processor.

Such machine-readable storage media may include, without limitation, non-transitory, tangible arrangements of articles manufactured or formed by a machine or device, including storage media such as hard discs, any other type of disk including floppy disks, optical disks, compact disc read-only memories (CD-ROMs), compact disc rewritables (CD-RWs), and magneto-optical disks, semiconductor devices such as ROMs, random access memories (RAMs) such as DRAMs, SRAMs, erasable programmable read-only memories (EPROMs), flash memories, electrically erasable programmable read-only memories (EEPROMs), PCM, magnetic or optical cards, or any other type of media suitable for storing electronic instructions.

Accordingly, some embodiments also include non-transitory, tangible machine-readable media containing instructions or containing design data, such as Hardware Description Language (HDL), which defines structures, circuits, apparatuses, processors and/or system features described herein. Such embodiments may also be referred to as program products.

Emulation (Including Binary Translation, Code Morphing, Etc.)

In some cases, an instruction converter may be used to convert an instruction from a source instruction set to a target instruction set. For example, the instruction converter may translate (e.g., using static binary translation or dynamic binary translation including dynamic compilation), morph, emulate, or otherwise convert an instruction to one or more other instructions to be processed by the core. The instruction converter may be implemented in software, hardware, firmware, or a combination thereof. The instruction converter may be on processor, off processor, or part on and part off processor.

The foregoing outlines features of one or more embodiments of the subject matter disclosed herein. These embodiments are provided to enable a person having ordinary skill in the art (PHOSITA) to better understand various aspects of the present disclosure. Certain well-understood terms, as well as underlying technologies and/or standards may be referenced without being described in detail. It is anticipated that the PHOSITA will possess or have access to background knowledge or information in those technologies and standards sufficient to practice the teachings of the present specification.

The PHOSITA will appreciate that they may readily use the present disclosure as a basis for designing or modifying other processes, structures, or variations for carrying out the same purposes and/or achieving the same advantages of the embodiments introduced herein. The PHOSITA will also recognize that such equivalent constructions do not depart from the spirit and scope of the present disclosure, and that they may make various changes, substitutions, and alterations herein without departing from the spirit and scope of the present disclosure.

In the foregoing description, certain aspects of some or all embodiments are described in greater detail than is strictly necessary for practicing the appended claims. These details are provided by way of nonlimiting example only, for the purpose of providing context and illustration of the disclosed embodiments. Such details should not be understood to be required, and should not be “read into” the claims as limitations. The phrase may refer to “an embodiment” or “embodiments.” These phrases, and any other references to embodiments, should be understood broadly to refer to any combination of one or more embodiments. Furthermore, the several features disclosed in a particular “embodiment” could just as well be spread across multiple embodiments. For example, if features 1 and 2 are disclosed in “an embodiment,” embodiment A may have feature 1 but lack feature 2, while embodiment B may have feature 2 but lack feature 1.

This specification may provide illustrations in a block diagram format, wherein certain features are disclosed in separate blocks. These should be understood broadly to disclose how various features interoperate, but are not intended to imply that those features must necessarily be embodied in separate hardware or software.

Furthermore, where a single block discloses more than one feature in the same block, those features need not necessarily be embodied in the same hardware and/or software. For example, a computer “memory” could in some circumstances be distributed or mapped between multiple levels of cache or local memory, main memory, battery-backed volatile memory, and various forms of persistent memory such as a hard disk, storage server, optical disk, tape drive, or similar. In certain embodiments, some of the components may be omitted or consolidated.

In a general sense, the arrangements depicted in the FIGURES may be more logical in their representations, whereas a physical architecture may include various permutations, combinations, and/or hybrids of these elements. Countless possible design configurations can be used to achieve the operational objectives outlined herein. Accordingly, the associated infrastructure has a myriad of substitute arrangements, design choices, device possibilities, hardware configurations, software implementations, and equipment options.

References may be made herein to a computer-readable medium, which may be a tangible and non-transitory computer-readable medium. As used in this specification and throughout the claims, a “computer-readable medium” should be understood to include one or more computer-readable mediums of the same or different types. A computer-readable medium may include, by way of nonlimiting example, an optical drive (e.g., CD/DVD/Blu-Ray), a hard drive, a solid state drive, a flash memory, or other nonvolatile medium. A computer-readable medium could also include a medium such as a ROM, a field-programmable gate array (FPGA) or ASIC configured to carry out the desired instructions, stored instructions for programming an FPGA or ASIC to carry out the desired instructions, an IP block that can be integrated in hardware into other circuits, or instructions encoded directly into hardware or microcode on a processor such as a microprocessor, DSP, microcontroller, or in any other suitable component, device, element, or object where appropriate and based on particular needs. A non-transitory storage medium herein is expressly intended to include any non-transitory special-purpose or programmable hardware configured to provide the disclosed operations, or to cause a processor to perform the disclosed operations.

Various elements may be “communicatively,” “electrically,” “mechanically,” or otherwise “coupled” to one another throughout this specification and the claims. Such coupling may be a direct, point-to-point coupling, or may include intermediary devices. For example, two devices may be communicatively coupled to one another via a controller that facilitates the communication. Devices may be electrically coupled to one another via intermediary devices such as signal boosters, voltage dividers, or buffers. Mechanically coupled devices may be indirectly mechanically coupled.

Any “module” or “engine” disclosed herein may refer to or include software, a software stack, a combination of hardware, firmware, and/or software, a circuit configured to carry out the function of the engine or module, or any computer-readable medium as disclosed above. Such modules or engines may, in appropriate circumstances, be provided on or in conjunction with a hardware platform, which may include hardware compute resources such as a processor, memory, storage, interconnects, networks and network interfaces, accelerators, or other suitable hardware. Such a hardware platform may be provided as a single monolithic device (e.g., in a PC form factor), or with some or part of the function being distributed (e.g., a “composite node” in a high-end data center, where compute, memory, storage, and other resources may be dynamically allocated and need not be local to one another).

There may be disclosed herein flow charts, signal flow diagram, or other illustrations showing operations being performed in a particular order. Unless otherwise expressly noted, or unless required in a particular context, the order should be understood to be a nonlimiting example only. Furthermore, in cases where one operation is shown to follow another, other intervening operations may also occur, which may be related or unrelated. Some operations may also be performed simultaneously or in parallel. In cases where an operation is said to be “based on” or “according to” another item or operation, this should be understood to imply that the operation is based at least partly on or according at least partly to the other item or operation. This should not be construed to imply that the operation is based solely or exclusively on, or solely or exclusively according to the item or operation.

All or part of any hardware element disclosed herein may readily be provided in an SoC, including a CPU package. An SoC represents an integrated circuit (IC) that integrates components of a computer or other electronic system into a single chip. Thus, for example, client devices or server devices may be provided, in whole or in part, in an SoC. The SoC may contain digital, analog, mixed-signal, and radio frequency functions, all of which may be provided on a single chip substrate. Other embodiments may include a multichip module (MCM), with a plurality of chips located within a single electronic package and configured to interact closely with each other through the electronic package.

In a general sense, any suitably-configured circuit or processor can execute any type of instructions associated with the data to achieve the operations detailed herein. Any processor disclosed herein could transform an element or an article (for example, data) from one state or thing to another state or thing. Furthermore, the information being tracked, sent, received, or stored in a processor could be provided in any database, register, table, cache, queue, control list, or storage structure, based on particular needs and implementations, all of which could be referenced in any suitable timeframe. Any of the memory or storage elements disclosed herein, should be construed as being encompassed within the broad terms “memory” and “storage,” as appropriate.

Computer program logic implementing all or part of the functionality described herein is embodied in various forms, including, but in no way limited to, a source code form, a computer executable form, machine instructions or microcode, programmable hardware, and various intermediate forms (for example, forms generated by an assembler, compiler, linker, or locator). In an example, source code includes a series of computer program instructions implemented in various programming languages, such as an object code, an assembly language, or a high level language such as OpenCL, FORTRAN, C, C++, JAVA, or HTML for use with various operating systems or operating environments, or in hardware description languages such as Spice, Verilog, and VHDL.

The source code may define and use various data structures and communication messages. The source code may be in a computer executable form (e.g., via an interpreter), or the source code may be converted (e.g., via a translator, assembler, or compiler) into a computer executable form, or converted to an intermediate form such as byte code. Where appropriate, any of the foregoing may be used to build or describe appropriate discrete or integrated circuits, whether sequential, combinatorial, state machines, or otherwise.

In one example embodiment, any number of electrical circuits of the FIGURES may be implemented on a board of an associated electronic device. The board can be a general circuit board that can hold various components of the internal electronic system of the electronic device and, further, provide connectors for other peripherals. Any suitable processor and memory can be suitably coupled to the board based on particular configuration needs, processing demands, and computing designs.

Note that with the numerous examples provided herein, interaction may be described in terms of two, three, four, or more electrical components. However, this has been done for purposes of clarity and example only. It should be appreciated that the system can be consolidated or reconfigured in any suitable manner. Along similar design alternatives, any of the illustrated components, modules, and elements of the FIGURES may be combined in various possible configurations, all of which are within the broad scope of this specification.

Numerous other changes, substitutions, variations, alterations, and modifications may be ascertained to one skilled in the art and it is intended that the present disclosure encompass all such changes, substitutions, variations, alterations, and modifications as falling within the scope of the appended claims. In order to assist the United States Patent and Trademark Office (USPTO) and, additionally, any readers of any patent issued on this application in interpreting the claims appended hereto, Applicant wishes to note that the Applicant: (a) does not intend any of the appended claims to invoke paragraph six (6) of 35 U.S.C. section 112 (pre-AIA) or paragraph (f) of the same section (post-AIA), as it exists on the date of the filing hereof unless the words “means for” or “steps for” are specifically used in the particular claims; and (b) does not intend, by any statement in the specification, to limit this disclosure in any way that is not otherwise expressly reflected in the appended claims.

### Example Implementations

There is disclosed in one example, a photoplethysmogram (PPG) sensor, comprising: a light-emitting diode (LED); a photodetector comprising an electrically-biased graphene metal junction having a conductor running therethrough; a current sensor to sense a current from the photodetector; and processing logic to compute a blood volume flow rate from the sensed current.

There is further disclosed an example PPG sensor, further comprising a low-pass filter to filter a time-invariant component from the sensed current.

There is further disclosed an example PPG sensor, wherein the conductor comprises a plurality of nanowires.

There is further disclosed an example PPG sensor, wherein the nanowires are silver nanowires.

There is further disclosed an example PPG sensor, wherein the photodetector overlays the LED.

There is further disclosed an example PPG sensor, wherein the LED is a green LED.

There is further disclosed an example PPG sensor, wherein the LED is not limited to any particular bandwidth.

There is further disclosed an example PPG sensor, further comprising an optical isolator around the photodetector.

There is further disclosed an example PPG sensor, further comprising an opaque encasement.

There is further disclosed an example PPG sensor, further comprising a transparent protective surface having a surface area to be substantially covered by a human subject's finger.

There is further disclosed an example PPG sensor, wherein the transparent protective surface is glass.

There is further disclosed a portable PPG sensor comprising the PPG sensor of a number of the above examples, a pulse meter, and a digital combined pulse and oxygenation display.

There is also disclosed an example computing apparatus, comprising: a hardware platform comprising a processor and a memory; a human interface device comprising a built-in oximeter, the built-in oximeter comprising a light-emitting diode (LED) and a photodetector comprising an electrically-biased graphene layer; and logic to receive blood flow rate and oxygen saturation data from the oximeter, and to alter a function of the computing apparatus according to the blood flow rate and oxygen saturation data.

There is further disclosed an example computing apparatus, wherein the human interface device is a trackpad.

There is further disclosed an example computing apparatus, wherein the human interface device is a mouse.

There is further disclosed an example computing apparatus, wherein the human interface device comprises a keyboard.

There is further disclosed an example computing apparatus, wherein the oximeter is built-in to a key.

There is further disclosed an example computing apparatus, wherein the key is a key commonly used for gaming operations.

There is further disclosed an example computing apparatus, wherein the human interface device is a palm rest on an encasement.

There is further disclosed an example computing apparatus, further comprising a low-pass filter to filter a time-invariant component from the sensed current.

There is further disclosed an example computing apparatus, wherein the photodetector further comprises a conductor having a plurality of nanowires.

There is further disclosed an example computing apparatus, wherein the nanowires are silver nanowires.

There is further disclosed an example computing apparatus, wherein the photodetector overlays the LED.

There is further disclosed an example computing apparatus, wherein the LED is a green LED.

There is further disclosed an example computing apparatus, wherein the LED is not limited to any particular bandwidth.

There is further disclosed an example computing apparatus, further comprising an optical isolator around the photodetector.

There is further disclosed an example computing apparatus, further comprising an opaque encasement.

There is further disclosed an example computing apparatus, further comprising a transparent protective surface having a surface area to be substantially covered by a human subject's finger.

There is further disclosed an example computing apparatus, wherein the transparent protective surface is glass or plastic.

There is further disclosed an example portable computing apparatus comprising the built-in oximeter of a number of the above examples, a pulse meter, and a digital combined pulse and oxygenation display.

There is also disclosed an example gaming laptop, comprising: a processor; a memory; a clamshell encasement comprising a lid and a base, the base comprising a keyboard, a trackpad, and an oximeter comprising a light-emitting diode (LED) and a photodetector comprising a graphene layer having a plurality of electrically-biased conductors passing therethrough; logic to compute a blood volume flow rate according to an induced current in the photo detector; and executable instructions within the memory to provide blood oxygen saturation data via an application programming interface (API).

There is further disclosed an example gaming laptop, further comprising a game encoded within the memory, the game including instructions to adjust the game according to the blood oxygen saturation data.

There is further disclosed an example gaming laptop, further comprising a low-pass filter to filter a time-invariant component from a sensed current.

There is further disclosed an example gaming laptop, wherein the electrically-biased conductors comprise a plurality of nanowires.

There is further disclosed an example gaming laptop, wherein the nanowires are silver nanowires.

There is further disclosed an example gaming laptop, wherein the photodetector overlays the LED.

There is further disclosed an example gaming laptop, wherein the LED is a green LED.

There is further disclosed an example gaming laptop, wherein the LED is not limited to any particular bandwidth.

There is further disclosed an example gaming laptop, further comprising an optical isolator around the photodetector.

There is further disclosed an example gaming laptop, further comprising an opaque encasement.

There is further disclosed an example gaming laptop, further comprising a transparent protective surface having a surface area to be substantially covered by a human subject's finger.

There is further disclosed an example gaming laptop, wherein the transparent protective surface is glass or plastic.

What is claimed is:

1. A photoplethysmogram (PPG) sensor, comprising:
a light-emitting diode (LED);
a photodetector comprising an electrically-biased graphene metal junction having a conductor running therethrough;
a current sensor to sense a current from the photodetector; and
processing logic to compute a blood volume flow rate from the sensed current.

2. The PPG sensor of claim 1, further comprising a low-pass filter to filter a time-invariant component from the sensed current.

3. The PPG sensor of claim 1, wherein the conductor comprises a plurality of nanowires.

4. The PPG sensor of claim 3, wherein the nanowires are silver nanowires.

5. The PPG sensor of claim 1, wherein the photodetector overlays the LED.

6. The PPG sensor of claim 1, wherein the LED is a green LED.

7. The PPG sensor of claim 1, further comprising an optical isolator around the photodetector.

8. The PPG sensor of claim 1, further comprising an opaque encasement.

9. The PPG sensor of claim 1, further comprising a transparent protective surface having a surface area to be substantially covered by a human subject's finger.

10. The PPG sensor of claim 9, wherein the transparent protective surface is glass.

11. A portable PPG sensor comprising the PPG sensor of claim 1, a pulse meter, and a digital combined pulse and oxygenation display.

12. A computing apparatus, comprising:
a hardware platform comprising a processor and a memory;
a human interface device comprising a built-in oximeter, the built-in oximeter comprising a light-emitting diode (LED) and a photodetector comprising an electrically-biased graphene layer; and
logic to receive blood flow rate and oxygen saturation data from the oximeter, and to alter a function of the computing apparatus according to the blood flow rate and oxygen saturation data.

13. The computing apparatus of claim 12, wherein the human interface device is a trackpad.

14. The computing apparatus of claim 12, wherein the human interface device is a mouse.

15. The computing apparatus of claim 12, wherein the human interface device comprises a keyboard.

16. The computing apparatus of claim 15, wherein the oximeter is built-in to a key.

17. The computing apparatus of claim 16, wherein the key is a key commonly used for gaming operations.

18. The computing apparatus of claim 12, wherein the human interface device is a palm rest on an encasement.

19. A gaming laptop, comprising:
a processor;
a memory;
a clamshell encasement comprising a lid and a base, the base comprising a keyboard, a trackpad, and an oximeter comprising a light-emitting diode (LED) and a photodetector comprising a graphene layer having a plurality of electrically-biased conductors passing therethrough;
logic to compute a blood volume flow rate and a pulse according to an induced current in the photo detector; and
executable instructions within the memory to provide blood oxygen saturation data and pulse data via an application programming interface (API).

20. The gaming laptop of claim 19, further comprising a game encoded within the memory, the game including instructions to adjust the game according to the blood oxygen saturation data or the pulse.
